(19) United States
(12) Patent Application Publication
(10) Pub. No.: US 2024/0407906 A1
(43) Pub. Date: Dec. 12, 2024

(54) REPLACEMENT HEART VALVE SYSTEM AND METHOD OF TREATING A NATIVE HEART VALVE

(71) Applicant: BOSTON SCIENTIFIC SCIMED, INC., MAPLE GROVE, MN (US)

(72) Inventors: James Holmberg, Champlin, MN (US); Zachary Klesch, Minneapolis, MN (US); Benjamin Mihelich, Minneapolis, MN (US); Todd Rowe, Excelsior, MN (US); Emily Dixon, Maple Grove, MN (US); Andrew John Mitterholzer, Eden Prairie, MN (US); Allison Michelle Wilcox, Minneapolis, MN (US); Soo-Young Yoon, Maple Grove, MN (US); Luis Gabriel Lopez-de-Victoria, Maple Grove, MN (US)

(73) Assignee: BOSTON SCIENTIFIC SCIMED, INC., MAPLE GROVE, MN (US)

(21) Appl. No.: 18/735,648

(22) Filed: Jun. 6, 2024

Related U.S. Application Data
(60) Provisional application No. 63/472,046, filed on Jun. 9, 2023.

(51) Int. Cl. A61F 2/01; A61F 2/24

(52) U.S. Cl. CPC A61F 2/013; A61F 2/0105; A61F 2/011; A61F 2/2433

(57) ABSTRACT

A replacement heart valve system may include a replacement heart valve implant including an expandable framework and a plurality of valve leaflets disposed within the expandable framework, and a filter element secured to the expandable framework and configured to be deployed downstream of the expandable framework, the filter element having a closed downstream end. A replacement heart valve system may include a filter element configured to be deployed across a native heart valve, wherein the filter element includes a lumen extending therethrough, and a replacement heart valve implant configured to be deployed within the lumen of the filter element. A method of treating a native heart valve may include deploying a filter element across the native heart valve, expanding an expandable balloon within the lumen of the filter element, and thereafter, deploying a replacement heart valve implant within the lumen of the filter element.

### CROSS-REFERENCE TO RELATED APPLICATIONS

This application claims the benefit of priority of U.S. Provisional Application No. 63/472,046 filed Jun. 9, 2023, the entire disclosure of which is hereby incorporated by reference.

### TECHNICAL FIELD

The disclosure relates generally to medical devices and more particularly to medical devices pertaining to replacement heart valve implants and procedures related thereto.

### BACKGROUND

Diseases and/or medical conditions that impact the cardiovascular system are prevalent throughout the world. Some relatively common medical conditions may include or be the result of inefficiency, ineffectiveness, or complete failure of one or more of the valves within the heart. Treatment of defective heart valves poses other challenges in that the treatment often requires the repair or outright replacement of the defective valve. Such therapies may be highly invasive to the patient. Disclosed herein are medical devices and/or procedures that may be used within a portion of the cardiovascular system in order to diagnose, treat, and/or repair the cardiovascular system and/or portions thereof. In some embodiments, one or more of the medical devices and/or procedures disclosed herein may be delivered and/or may be performed percutaneously and, thus, may be much less invasive to the patient, although other surgical methods and approaches may also be used. For example, in some embodiments, one or more of the medical devices and/or procedures disclosed herein may be delivered and/or may be performed apically. Of the known medical devices and methods, each has certain advantages and disadvantages. There is an ongoing need to provide alternative medical devices as well as alternative methods for manufacturing and using medical devices.

### SUMMARY

In one example, a replacement heart valve system may comprise a replacement heart valve implant comprising an expandable framework and a plurality of valve leaflets disposed within the expandable framework, and a filter element secured to the expandable framework and configured to be deployed downstream of the expandable framework, the filter element having a closed downstream end.

In addition or alternatively to any example described herein, the filter element is configured to be deployed before the replacement heart valve implant.

In addition or alternatively to any example described herein, the filter element is detachable from the expandable framework in situ.

In addition or alternatively to any example described herein, the filter element is retrievable upstream through the replacement heart valve implant while leaving the replacement heart valve implant in place.

In addition or alternatively to any example described herein, the filter element is retrievable downstream from the replacement heart valve implant while leaving the replacement heart valve implant in place.

In addition or alternatively to any example described herein, the filter element includes an open mouth at an upstream end, wherein the open mouth is spaced apart from the expandable framework.

In addition or alternatively to any example described herein, filter element is configured to capture particulates while permitting blood to pass through the filter element.

In addition or alternatively to any example described herein, the replacement heart valve system may comprise a delivery sheath configured to transport the replacement heart valve implant and the filter element to a position upstream of a native heart valve.

In addition or alternatively to any example described herein, a replacement heart valve system may comprise a filter element configured to be deployed across a native heart valve, wherein the filter element includes a lumen extending therethrough from a first end to a second end, and a replacement heart valve implant comprising an expandable framework and a plurality of valve leaflets disposed within the expandable framework. The replacement heart valve implant may be configured to be deployed within the lumen of the filter element.

In addition or alternatively to any example described herein, the filter includes a medial portion having an hourglass shape in a deployed configuration.

In addition or alternatively to any example described herein, the hourglass shape extends from the first end of the filter element to the second end of the filter element.

In addition or alternatively to any example described herein, the first end includes a first sealing element configured to engage native tissue upstream of the native heart valve and the second end includes a second sealing element configured to engage native tissue downstream of the native heart valve.

In addition or alternatively to any example described herein, the replacement heart valve system may comprise an expandable balloon disposed proximate a distal end of a balloon catheter, wherein the expandable balloon is configured to be expanded within the lumen of the filter element before deploying the replacement heart valve implant within the lumen of the filter element.

In addition or alternatively to any example described herein, the filter element is permeable to blood.

In addition or alternatively to any example described herein, a method of treating a native heart valve may comprise: deploying a filter element across the native heart valve, wherein the filter element includes a lumen extending from a first end of the filter element to a second end of the filter element; expanding an expandable balloon within the lumen of the filter element; and thereafter, deploying a replacement heart valve implant within the lumen of the filter element.

In addition or alternatively to any example described herein, the filter element is configured to capture and retain debris dislodged while expanding the expandable balloon within the lumen of the filter element.

In addition or alternatively to any example described herein, the filter element includes a mesh extending from the first end to the second end.

In addition or alternatively to any example described herein, the filter element includes a permeable membrane extending from the first end to the second end.

In addition or alternatively to any example described herein, expanding the expandable balloon within the lumen of the filter element includes urging the filter element radially outward against an annulus of the native heart valve.

In addition or alternatively to any example described herein, prior to deploying the replacement heart valve implant within the lumen of the filter element, the filter element permits native valve leaflets of the native heart valve to at least partially close the native heart valve.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partial cutaway view illustrating selected aspects of a replacement heart valve system positioned within a native heart valve of a heart;

FIG. 2 illustrates a filter element of the replacement heart valve system of FIG. 1 capturing debris;

FIGS. 3-4 illustrate selected aspects related to the filter element of FIGS. 1-2 being retrieved apically; and

FIGS. 5-6 illustrate selected aspects related to the filter element of FIGS. 1-2 being retrieved percutaneously;

FIG. 7 is a partial cutaway view illustrating a filter element of a replacement heart valve system positioned within a native heart valve of a heart;

FIG. 8 illustrates the native valve leaflets of the native heart valve closed with the filter element of FIG. 7 positioned within the native heart valve;

FIG. 9 illustrates selected aspects of an expandable balloon disposed within the filter element of FIGS. 7-8; and

FIG. 10 illustrates selected aspects of a replacement heart valve implant disposed within the filter element of FIGS. 7-9.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

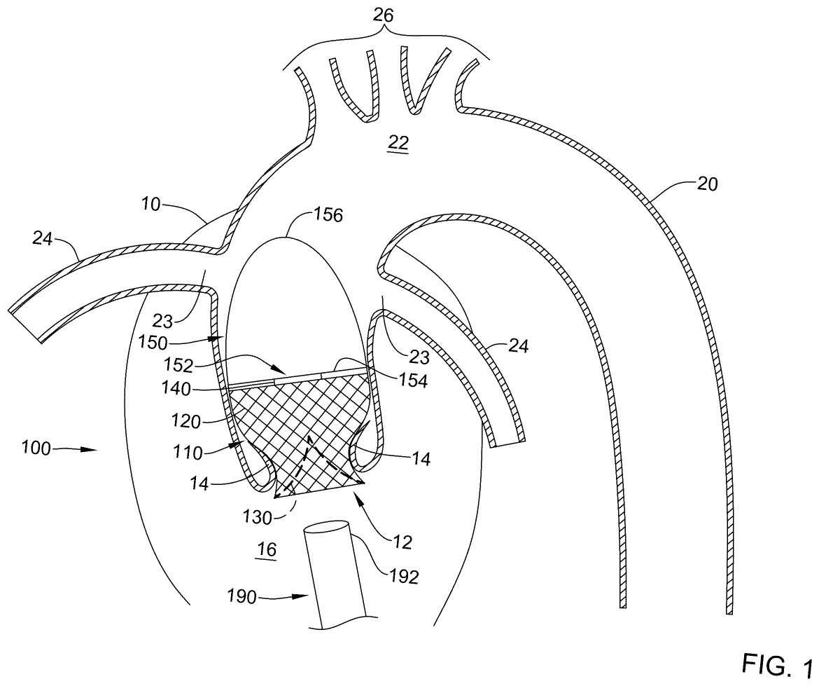

FIG. 1 illustrates a schematic partial cut-away view of a portion of a patient's heart 10 including a native heart valve (e.g., the aortic valve 12) having native valve leaflets 14 disposed within and/or extending from the native valve annulus, the left ventricle 16, and certain connected vasculature, such as the aorta 20 connected to the native heart valve (e.g., the aortic valve 12) of the patient's heart 10 by the aortic arch 22 and the ascending aorta, the coronary ostia 23 of the coronary arteries 24, which extend from the aortic sinuses and/or the ascending aorta, and other large arteries 26 (e.g., subclavian and/or carotid arteries, etc.) that extend from the aortic arch 22 to important internal organs. For the purpose of this disclosure, the discussion herein is generally directed toward treating the aortic valve 12 and will be so described in the interest of brevity. This, however, is not intended to be limiting as the skilled person will recognize that the following discussion may also apply to other heart valves, vessels, and/or treatment locations within the patient.

FIG. 1 also schematically illustrates selected aspects of a replacement heart valve system 100 positioned within the native heart valve (e.g., the aortic valve 12). In at least some embodiments, the replacement heart valve system 100 may comprise a replacement heart valve implant 110 and a filter element 150. In some embodiments, the replacement heart valve system 100 may comprise a delivery sheath 190 configured to transport the replacement heart valve implant 110 and the filter element 150 to a position adjacent the native heart valve (e.g., the aortic valve 12). In some embodiments, the delivery sheath 190 may be configured to transport the replacement heart valve implant 110 and the filter element 150 to a position upstream of the native heart valve (e.g., the aortic valve 12). In some embodiments, the delivery sheath 190 may be configured to make an apical approach to the native heart valve (e.g., the aortic valve 12). For example, FIG. 1 schematically illustrates the delivery sheath 190 extending through and/or from an apex of the heart 10 toward the native heart valve (e.g., the aortic valve 12) such that a distal end 192 of the delivery sheath 190 is a downstreammost extent of the delivery sheath 190 within the patient and/or the heart 10.

It should be appreciated that the replacement heart valve implant 110 can be any type of replacement heart valve (e.g., a mitral valve, an aortic valve, etc.). In use, the replacement heart valve implant 110 may be implanted (e.g., such as through transcatheter delivery) in the native heart valve (e.g., the aortic valve 12) of the heart 10. The replacement heart valve implant 110 may be configured to allow one-way flow through the replacement heart valve implant 110 from an inflow end to an outflow end.

The replacement heart valve implant 110 may include an expandable framework 120 defining a central lumen. Some suitable but non-limiting examples of materials that may be used to form the expandable framework 120, including but not limited to metals and metal alloys, composites, ceramics, polymers, and the like, are described below. The replacement heart valve implant 110 and/or the expandable framework 120 may be configured to shift between a collapsed configuration and an expanded configuration. In some embodiments, the expandable framework 120 may be self-expanding. In some embodiments, the expandable framework 120 may be self-biased toward the expanded configuration. In some embodiments, the expandable framework 120 may be mechanically expandable. In some embodiments, the expandable framework 120 may be balloon expandable.

In some embodiments, the replacement heart valve implant 110 may include a plurality of valve leaflets 130 disposed within the central lumen of the expandable framework 120. The plurality of valve leaflets 130 may be coupled, secured, and/or fixedly attached to the expandable framework 120 at a plurality of commissures. The plurality of valve leaflets 130 may be configured to shift between an open position and a closed position. The plurality of valve leaflets 130 may be configured to substantially restrict fluid flow through the replacement heart valve implant 110 in the closed position. The plurality of valve leaflets 130 may move apart and/or may be spaced apart from each other in the open position to permit fluid flow through the replacement heart valve implant 110.

In some embodiments, the plurality of valve leaflets 130 may be comprised of a polymer, such as a thermoplastic polymer. In some embodiments, the plurality of valve leaflets 130 may include at least 50 percent by weight of a polymer. In some embodiments, the plurality of valve leaflets 130 may be formed from bovine pericardial or other living tissue. Other configurations and/or materials are also contemplated.

In some embodiments, the filter element 150 may be secured to the expandable framework 120. In some embodiments, the filter element 150 may be fixedly secured and/or fixedly attached to the expandable framework 120. In some embodiments, the filter element 150 may be configured to be deployed downstream of the replacement heart valve implant 110 and/or the expandable framework 120.

The filter element 150 may include an open mouth 152 at an upstream end. In some embodiments, the open mouth 152 may be supported by and/or may be defined by a support hoop. In some embodiments, the filter element 150 and/or the open mouth 152 may be self-supporting. In some embodiments, the open mouth 152 may include and/or may be defined by a cinch element 154, such as a filament, a thread, a suture, or a wire. The cinch element 154 may be configured to shift between an open configuration and a closed configuration. When the filter element 150 is in a deployed configuration, the cinch element 154 may be in the open configuration.

The filter element 150 may include a closed downstream end 156. In some embodiments, the filter element 150 may be formed as and/or from a mesh. In some embodiments, the filter element 150 may formed as and/or from a porous membrane. In some embodiments, the filter element 150 may formed as and/or from a permeable membrane. In some embodiments, the filter element 150 may have a plurality of openings and/or a plurality of pores formed therein. In some embodiments, the plurality of openings and/or the plurality of pores may be generally round in shape and/or cross-section. In some embodiments, the plurality of openings and/or the plurality of pores may have a size ranging from about 50 microns to about 250 microns in diameter. In some embodiments, the plurality of openings and/or the plurality of pores may be irregularly shaped and/or may vary in size. Other configurations are also contemplated. Some examples of suitable but non-limiting materials for the filter element 150, including but not limited to metallic materials and/or polymeric materials, are described below.

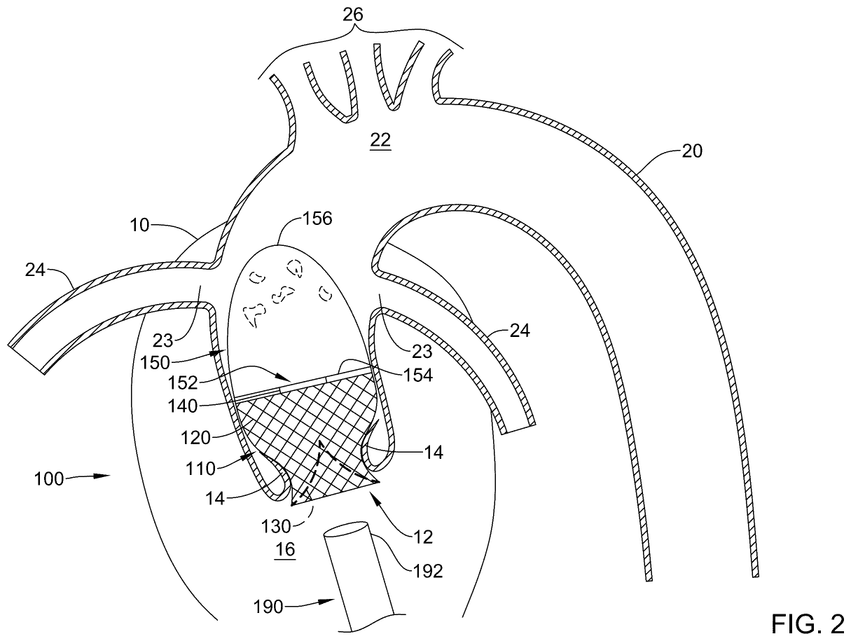

In some embodiments, the filter element 150 may be configured to capture particulates and/or debris (e.g., thrombus, calcification, etc.) while permitting blood to pass through the filter element 150, as shown schematically in FIG. 2. In at least some embodiments, all blood or fluid entering and/or passing through the open mouth 152 passes through the filter element 150. In some embodiments, all blood or fluid entering and/or passing through the open mouth 152 must pass through the filter element 150.

Returning briefly to FIG. 1, in some embodiments, the filter element 150 may be secured to the expandable framework 120 of the replacement heart valve implant 110 by and/or using at least one tether 140. In some embodiments, the filter element 150 may be fixedly attached to the expandable framework 120 of the replacement heart valve implant 110 by and/or using the at least one tether 140. In some embodiments, the at least one tether 140 may be formed from wire(s), suture(s), thread(s), filament(s), etc. In some alternative configurations, the filter element 150 may be integrally formed with the replacement heart valve implant 110 and/or the expandable framework 120.

In some embodiments, the filter element 150 and/or the open mouth 152 may be spaced apart from the expandable framework 120 of the replacement heart valve implant 110. In some embodiments, the filter element 150 and/or the open mouth 152 may be spaced apart from the expandable framework 120 of the replacement heart valve implant 110 by about 0 mm to about 100 mm. In some embodiments, the filter element 150 and/or the open mouth 152 may be spaced apart from the expandable framework 120 of the replacement heart valve implant 110 by about 0 mm to about 50 mm. Other configurations and/or spacing are also contemplated.

In at least some embodiments, the filter element 150 may be configured to be deployed before the replacement heart valve implant 110. For example, the filter element 150 may be disposed distal of the replacement heart valve implant 110 within the delivery sheath 190. In some embodiments, the filter element 150 and the replacement heart valve implant 110 may be deployed from the delivery sheath 190 via proximal movement of the delivery sheath 190 relative to the filter element 150 and the replacement heart valve implant 110. In some embodiments, the filter element 150 and the replacement heart valve implant 110 may be advanced distally out of the delivery sheath 190. Other configurations are also contemplated.

In some embodiments, deploying the filter element 150 before the replacement heart valve implant 110 may permit the filter element 150 to capture any debris, particulates, calcification, thrombus, etc. that may be dislodged during deployment and/or expansion of the replacement heart valve implant 110 within the native heart valve (e.g., the aortic valve 12).

In some embodiments, the filter element 150 may be configured to be permanently left within the patient and/or attached to the replacement heart valve implant 110 and/or the expandable framework 120. For the purpose of this disclosure, “permanently” may be considered to include an extended period time that may last for weeks, months, or years. In some embodiments, the filter element 150 may never need to be removed from the patient, depending upon the debris load within the filter element 150. If at some point in time a practitioner determines that the debris load within the filter element 150 is too great, and/or poses unnecessary and/or unacceptable risk to the patient, the filter element 150 may be detachable from the replacement heart valve implant 110 and/or the expandable framework 120 in situ.

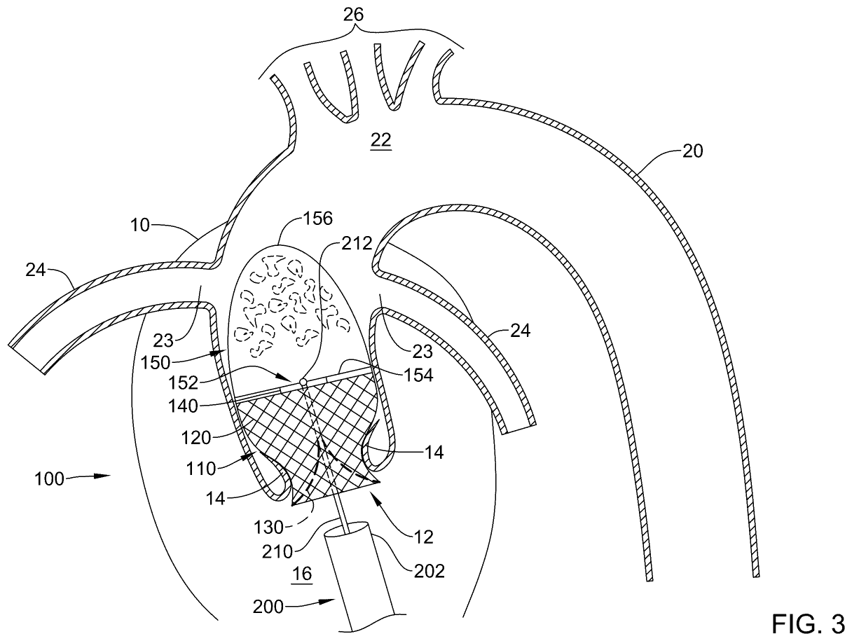

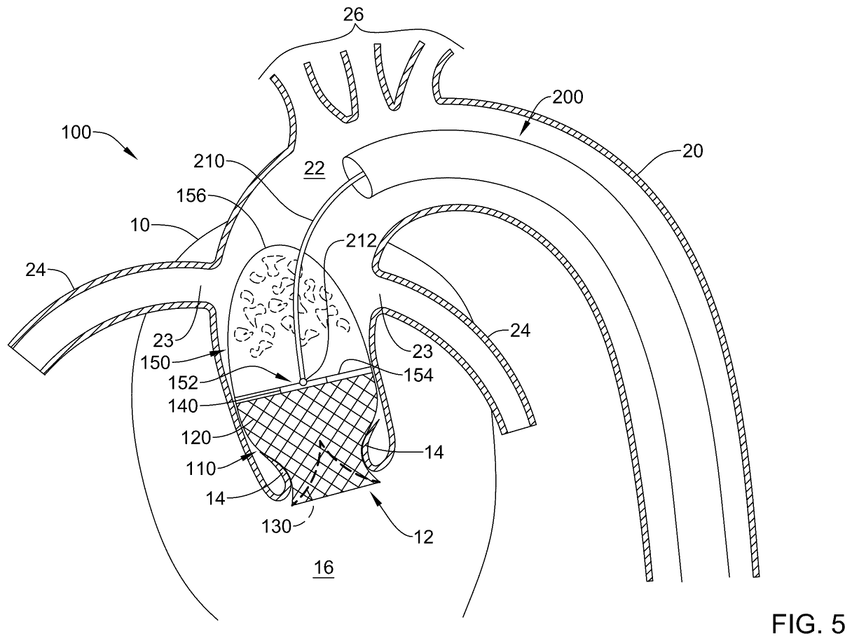

FIGS. 3-6 illustrate selected aspects related to retrieving the filter element 150 in situ. In some embodiments, the replacement heart valve system 100 may include a retrieval catheter 200. The retrieval catheter 200 may be advanced to a position adjacent the replacement heart valve implant 110 and/or the filter element 150, as seen in FIGS. 3 and 5. In some embodiments, the retrieval catheter 200 may be advanced to a position upstream of the replacement heart valve implant 110 and/or the filter element 150, as seen in FIG. 3. In some embodiments, the retrieval catheter 200 may be advanced to a position downstream of the replacement heart valve implant 110 and/or the filter element 150, as seen in FIG. 5.

In some embodiments, the retrieval catheter 200 may be the delivery sheath 190 and retrieval may occur during and/or shortly after implantation of the replacement heart valve implant 110 and the filter element 150. In some embodiments, the retrieval catheter 200 may be separate and/or different from the delivery sheath 190 and retrieval may occur after some period of time beyond the implantation procedure of the replacement heart valve implant 110 and the filter element 150.

A filter retrieval tool 210 may be slidably disposed within the retrieval catheter 200. The filter retrieval tool 210 may be advanceable distally out of a distal end 202 of the retrieval catheter 200. The filter retrieval tool 210 may be advanceable distally out of the distal end 202 of the retrieval catheter 200 toward and/or to the open mouth 152 and/or the cinch element 154 of the filter element 150, as seen in FIGS. 3 and 5.

In some embodiments, the filter retrieval tool 210 may be configured to grasp the filter element 150 and/or the cinch element 154. In some embodiments, the filter retrieval tool 210 may include a distal clamping element 212 or other structure disposed at and/or adjacent to a distal end of the filter retrieval tool 210. The distal clamping element 212 may be configured to grasp the filter element 150 and/or the cinch element 154. In some alternative configurations, the filter retrieval tool 210 may include a distal element configured to engage the filter element 150 and/or the cinch element 154 that is different from the distal clamping element 212, and/or the distal clamping element 212 may take various forms. For example, the filter retrieval tool 210 may include a hook, a pair of jaws, a clamshell structure, etc. Other configurations are also contemplated.

In some embodiments, the replacement heart valve system 100 may further comprise a cutting tool (not shown) slidably disposed within the retrieval catheter 200. In some embodiments, the cutting tool may be integrally formed with the filter retrieval tool 210. In some embodiments, the cutting tool may be the filter retrieval tool 210 and/or a cutting element disposed on the filter retrieval tool 210. In some embodiments, the cutting tool may be separate from the filter retrieval tool 210.

In some embodiments, the at least one tether 140 may be severed and/or cut in situ to detach the filter element 150 from the replacement heart valve implant 110 and/or the expandable framework 120 in situ. This may permit the replacement heart valve implant 110 to remain in place in the event the filter element 150 needs to be removed.

In some embodiments, the cinch element 154 may be pulled and/or tightened by the filter retrieval tool 210 and/or the distal clamping element 212 to shift the cinch element 154 from the open configuration to the closed configuration. In the closed configuration, any debris, particulates, calcification, thrombus, etc. disposed within the filter element 150 will be substantially contained and/or retained therein. In some embodiments, the cinch element 154 may be pulled and/or tightened before severing and/or cutting the at least one tether 140. In some embodiments, the at least one tether 140 may be severed and/or cut before the cinch element 154 is pulled and/or tightened. In some embodiments, the at least one tether 140 may be severed or cut while pulling and/or tightening the cinch element 154.

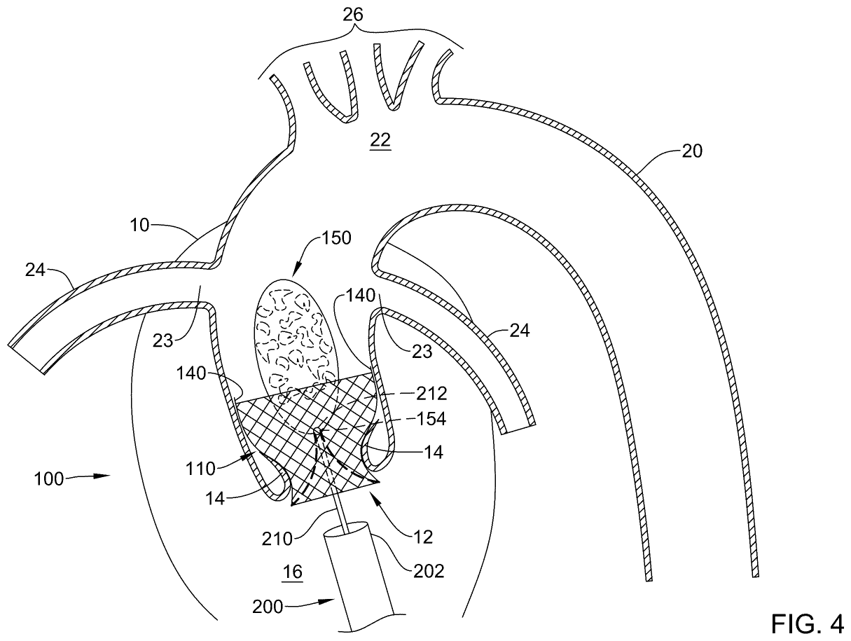

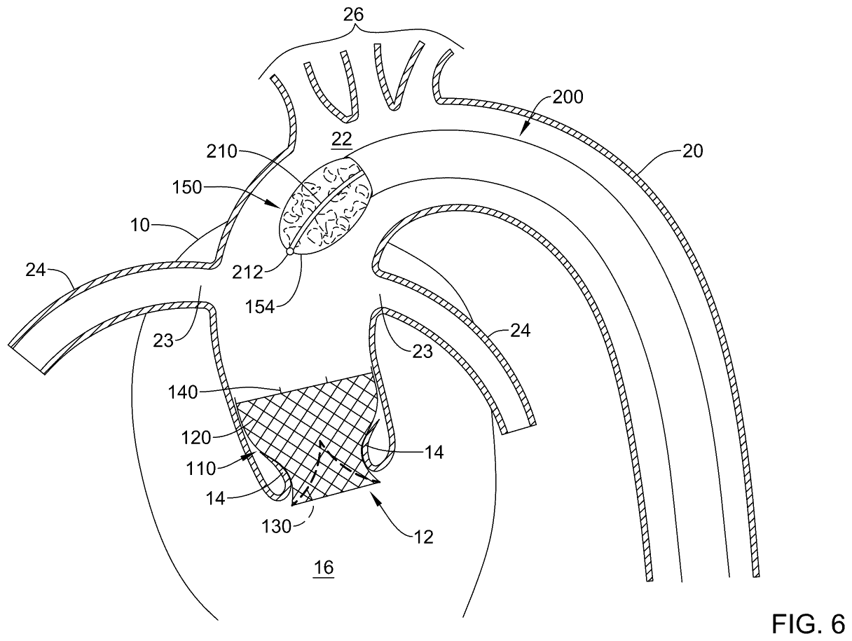

In some embodiments, after pulling and/or tightening the cinch element 154 and severing and/or cutting the at least one tether 140, the filter element 150 may be retrievable upstream through the replacement heart valve implant 110 while leaving the replacement heart valve implant 110 in place within the native heart valve (e.g., the aortic valve 12), as seen in FIG. 4. In some embodiments, after pulling and/or tightening the cinch element 154 and severing and/or cutting the at least one tether 140, the filter element 150 may be retrievable downstream from the replacement heart valve implant 110 while leaving the replacement heart valve implant 110 in place within the native heart valve (e.g., the aortic valve 12), as seen in FIG. 6.

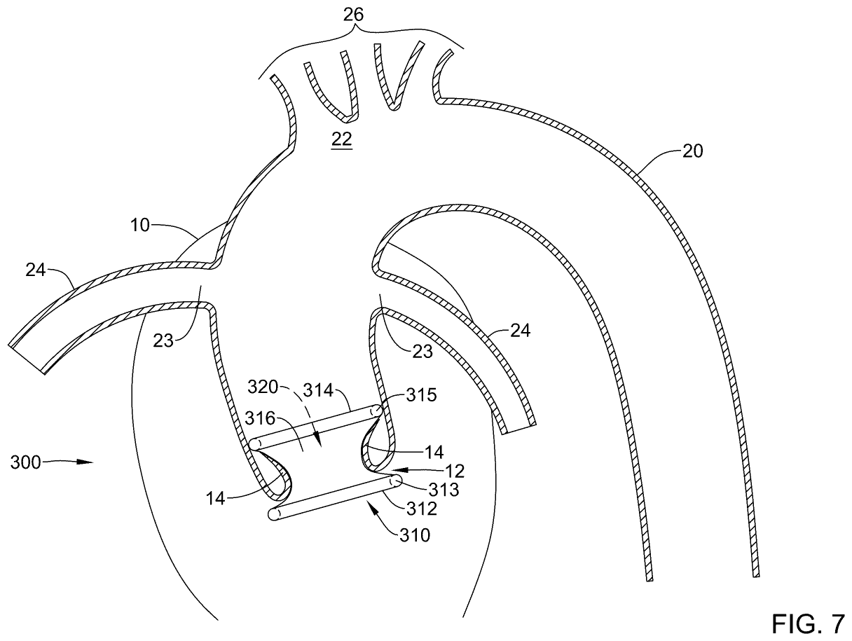

FIGS. 7-10 illustrates selected aspects related to a replacement heart valve system 300. It should be noted that all elements of the replacement heart valve system 300 are not shown in any single figure in the interest of clarity and the figures and description related to the replacement heart valve system 300 should be considered in aggregate.

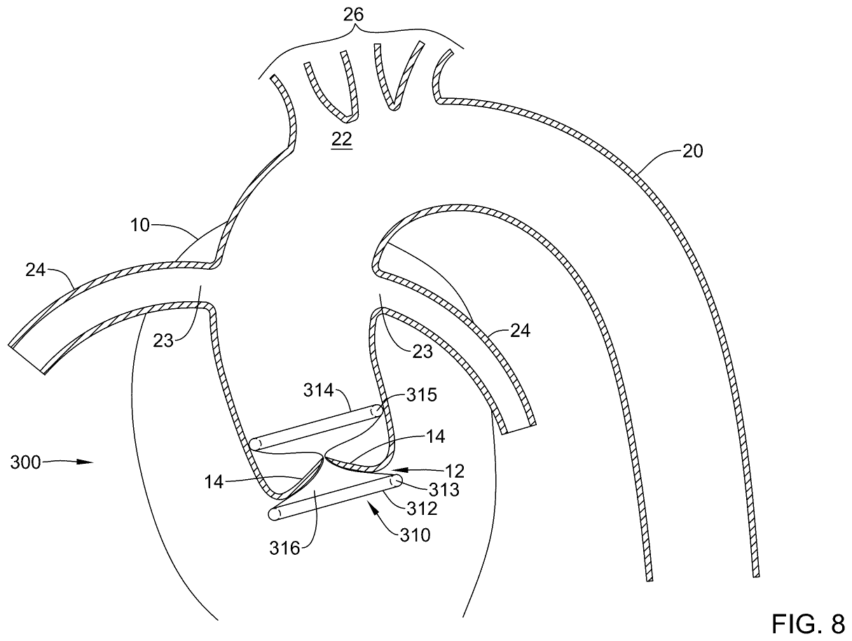

The replacement heart valve system 300 may include a filter element 310 configured to be deployed across and/or within a native heart valve (e.g., the aortic valve 12), as seen in FIGS. 7-8. In some embodiments, the filter element 310 may comprise a first end 312, a second end 314, and a lumen 320 extending therethrough from the first end 312 to the second end 314. In some embodiments, the first end 312 may be an upstream end and the second end 314 may be a downstream end. In some alternative embodiments, the first end 312 may be the downstream end and the second end 314 may be the upstream end.

The filter element 310 may be configured to shift between a delivery configuration (e.g., when disposed within a delivery catheter) and a deployed configuration (e.g., when deployed within the native heart valve). The first end 312 of the filter element 310 may include a first sealing element 313 configured to engage native tissue upstream of the native heart valve (e.g., the aortic valve 12) and/or the native valve leaflets 14 and a second sealing element 315 configured to engage native tissue downstream of the native heart valve (e.g., the aortic valve 12) and/or the native valve leaflets 14.

In some embodiments, the first sealing element 313 may include a first expandable annular ring. In some embodiments, the first sealing element 313 and/or the first expandable annular ring may be inflatable and/or hollow. In some embodiments, the first sealing element 313 and/or the first expandable annular ring may be in fluid communication with a source of inflation fluid. In some embodiments, the inflation fluid may be a liquid, a gas, a gel, a foam, etc. that is biocompatible. In some embodiments, the inflation fluid may be transported into the first sealing element 313 and/or the first expandable annular ring to fill and/or expand the first sealing element 313 and/or the first expandable annular ring to a deployed state in situ. In the deployed state, the first sealing element 313 and/or the first expandable annular ring may exert a radially outward force against the native tissue upstream of the native heart valve (e.g., the aortic valve 12) and/or the native valve leaflets 14.

In some embodiments, the second sealing element 315 may include a second expandable annular ring. In some embodiments, the second sealing element 315 and/or the second expandable annular ring may be inflatable and/or hollow. In some embodiments, the second sealing element 315 and/or the second expandable annular ring may be in fluid communication with a source of inflation fluid. In some embodiments, the inflation fluid may be a liquid, a gas, a gel, a foam, etc. that is biocompatible. In some embodiments, the inflation fluid may be transported into the second sealing element 315 and/or the second expandable annular ring to fill and/or expand the second sealing element 315 and/or the second expandable annular ring to a deployed state in situ. In the deployed state, the second sealing element 315 and/or the second expandable annular ring may exert a radially outward force against the native tissue downstream of the native heart valve (e.g., the aortic valve 12) and/or the native valve leaflets 14.

The filter element 310 may include a medial portion 316 having an hourglass shape in the deployed configuration. In at least some embodiments, the hourglass shape may extend from the first end 312, the first sealing element 313, and/or the first expandable annular ring to the second end 314, the second sealing element 315 and/or the second expandable annular ring. The lumen 320 may extend axially within and/or through the medial portion 316.

In some embodiments, the filter element 310 and/or the medial portion 316 may be formed as and/or from a mesh. In some embodiments, the mesh may extend from the first end 312 and/or the first sealing element 313 to the second end 314 and/or the second sealing element 315. In some embodiments, the filter element 310 and/or the medial portion 316 may formed as and/or from a porous membrane. In some embodiments, the porous membrane may extend from the first end 312 and/or the first sealing element 313 to the second end 314 and/or the second sealing element 315. In some embodiments, the filter element 310 and/or the medial portion 316 may formed as and/or from a permeable membrane. In some embodiments, the permeable membrane may extend from the first end 312 and/or the first sealing element 313 to the second end 314 and/or the second sealing element 315. In some embodiments, the filter element 310 and/or the medial portion 316 may have a plurality of openings and/or a plurality of pores formed therein. In some embodiments, the plurality of openings and/or the plurality of pores may be generally round in shape and/or cross-section. In some embodiments, the plurality of openings and/or the plurality of pores may have a size ranging from about 50 microns to about 250 microns in diameter. In some embodiments, the plurality of openings and/or the plurality of pores may be irregularly shaped and/or may vary in size. Other configurations are also contemplated. Some examples of suitable but non-limiting materials for the filter element 310 and/or the medial portion 316, including but not limited to metallic materials and/or polymeric materials, are described below.

In some embodiments, the filter element 310 and/or the medial portion 316 may be configured to capture and/or trap particulates and/or debris (e.g., thrombus, calcification, etc.) between the filter element 310 and/or the medial portion 316 and the native tissue (e.g., the aortic sinus, etc.) while permitting blood to pass through the filter element 310 and/or the medial portion 316. Accordingly, the filter element 310 and/or the medial portion 316 may be permeable to blood. As will be apparent, the filter element 310 may be particularly useful during a balloon aortic valvuloplasty (BAV) procedure prior to implantation of a replacement heart valve implant since the filter element 310 may be configured to trap particulates and/or debris (e.g., thrombus, calcification, etc.) released and/or dislodged from the native valve leaflets 14 during the BAV procedure, thereby preventing the particulates and/or debris (e.g., thrombus, calcification, etc.) from transiting downstream into smaller blood vessels.

In some embodiments, the filter element 310 and/or the medial portion 316 may be flexible and/or elastic. In some embodiments, the filter element 310 and/or the medial portion 316 may permit the native valve leaflets 14 to temporarily open and close, at least partially, to maintain and/or provide at least partial function of the native heart valve (e.g., the aortic valve 12) during the procedure, as shown in FIGS. 7-8.

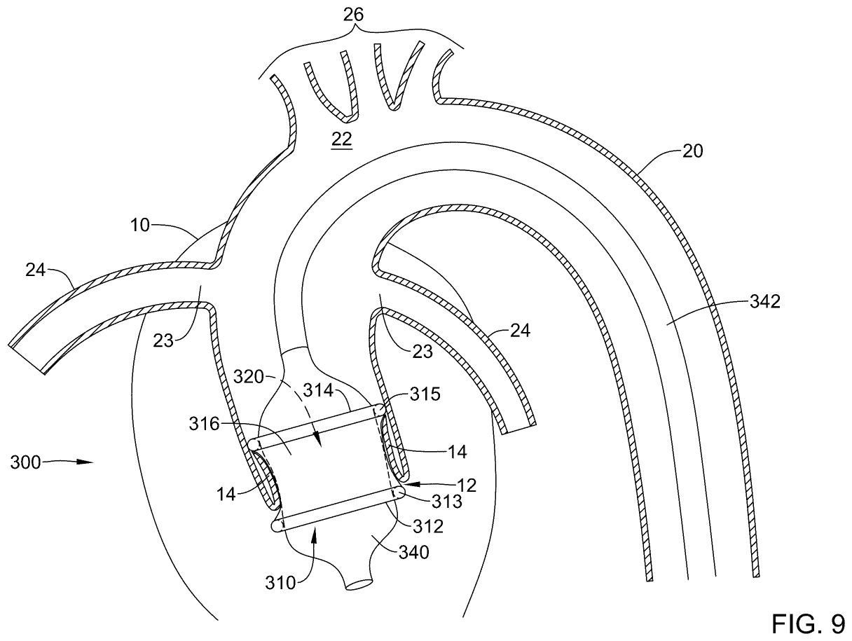

In some embodiments, the replacement heart valve system 300 may include an expandable balloon 340 disposed proximate a distal end of a balloon catheter 342. In at least some embodiments, the balloon catheter 342 may be advanced percutaneously to the native heart valve (e.g., the aortic valve 12), as seen in FIG. 9. The expandable balloon 340 may be in fluid communication with a source of inflation fluid (not shown) via the balloon catheter 342. In some embodiments, the expandable balloon 340 may be configured to be expanded within the lumen 320 of the filter element 310.

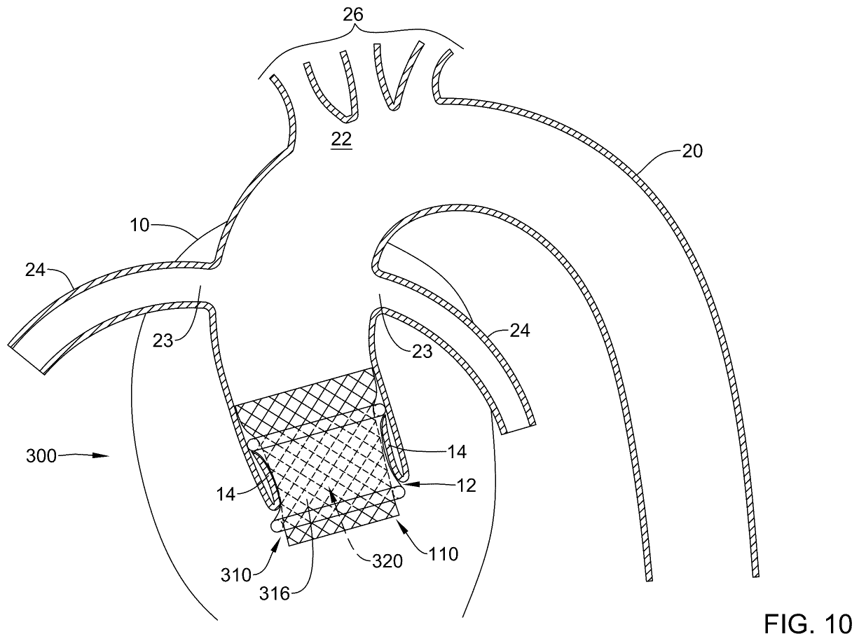

The replacement heart valve system 300 may include a replacement heart valve implant 110, as described herein. The replacement heart valve implant 110 may be configured to be deployed within the lumen 320 of the filter element 310, as shown in FIG. 10.

In some embodiments, the expandable balloon 340 may be configured to be expanded within the lumen 320 of the filter element 310 before the replacement heart valve implant 110, such as during a BAV procedure. When performing the BAV procedure, the expandable balloon 340 may be configured to apply radially outward force to the native valve leaflets 14 to break and/or dislodge calcification that may have formed thereon. Doing so may sometimes cause particulates and/or debris (e.g., thrombus, calcification, etc.) to be released into the blood stream, which may increase risk to the patient. The filter element 310 may be configured to be placed and/or deployed across and/or within the native heart valve (e.g., the aortic valve 12) prior to performing the BAV procedure to trap particulates and/or debris (e.g., thrombus, calcification, etc.) and thereby prevent the particulates and/or debris (e.g., thrombus, calcification, etc.) from being released into the blood stream.

In some embodiments, the replacement heart valve system 300 may comprise a delivery device (not shown) configured to transport the replacement heart valve implant 110 and/or the filter element 310 to a position adjacent the native heart valve (e.g., the aortic valve 12). In some embodiments, the delivery device may be configured to transport the replacement heart valve implant 110 and/or the filter element 310 to a position upstream of the native heart valve (e.g., the aortic valve 12). In some embodiments, the delivery device may be configured to make an apical approach to the native heart valve (e.g., the aortic valve 12). In some embodiments, the delivery device may be configured to transport the replacement heart valve implant 110 and/or the filter element 310 to a position downstream of the native heart valve (e.g., the aortic valve 12). In some embodiments, the delivery device may be configured to make a percutaneous approach and/or an aortic approach to the native heart valve (e.g., the aortic valve 12).

A method of treating a native heart valve (e.g., the aortic valve 12) may comprise deploying the filter element 310 across the native heart valve (e.g., the aortic valve 12), as seen in FIG. 7. In some embodiments, the method may comprise advancing a delivery device to a position adjacent the native heart valve (e.g., the aortic valve 12) prior to deploying the filter element 310. In some embodiments, the method may comprise deploying the filter element 310 from the delivery device within and/or across the native heart valve (e.g., the aortic valve 12).

In some embodiments, the method may comprise expanding the first sealing element 313 and/or the first expandable annular ring against native tissue upstream of the native heart valve (e.g., the aortic valve 12) and/or the native valve leaflets 14. In some embodiments, the method may comprise expanding the second sealing element 315 and/or the second expandable annular ring against native tissue downstream of the native heart valve (e.g., the aortic valve 12) and/or the native valve leaflets 14. In some embodiments, the method may comprise trapping and/or containing the native valve leaflets 14 radially outward of a medial portion 316 of the filter element 310 and radially inward of the native tissue (e.g., an annulus of the native heart valve and/or a sinus of the native heart valve), and between the first sealing element 313 and/or the first expandable annular ring and the second sealing element 315 and/or the second expandable annular ring.

In some embodiments, the method may comprise advancing a balloon catheter 342 to a position proximate the native heart valve (e.g., the aortic valve 12). In some embodiments, the method may comprise positioning the expandable balloon 340 within the native heart valve (e.g., the aortic valve 12). In some embodiments, the method may comprise expanding the expandable balloon 340 within the lumen 320 of the filter element 310. In some embodiments, the method may comprise expanding the expandable balloon 340 within the lumen 320 of the filter element 310 within the native heart valve (e.g., the aortic valve 12), as seen in FIG. 9. In some embodiments, expanding the expandable balloon 340 within the lumen 320 of the filter element 31 may include urging the filter element 310 and/or the medial portion 316 of the filter element 310 radially outward against an annulus of the native heart valve (e.g., the aortic valve 12). The filter element 310 may be configured to capture and retain particulates and/or debris (e.g., thrombus, calcification, etc.) dislodged while expanding the expandable balloon 340 within the lumen 320 of the filter element 310.

In some embodiments, the method may comprise thereafter, deploying the replacement heart valve implant 110 within the lumen 320 of the filter element 310, as seen in FIG. 10. In some embodiments, prior to deploying the replacement heart valve implant 110 within the lumen 320 of the filter element 310, the filter element 310 may permit the native valve leaflets 14 of the native heart valve (e.g., the aortic valve 12) to at least partially close the native heart valve (e.g., the aortic valve 12), as seen in FIG. 8.

In some embodiments, the method may comprise after deploying the replacement heart valve implant 110 within the lumen 320 of the filter element 310, leaving the filter element 310 permanently in place within the native heart valve (e.g., the aortic valve 12).

The materials that can be used for the various components of the system and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the delivery device, the replacement heart valve implant, the balloon catheter, the filter element, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A replacement heart valve system, comprising:
a replacement heart valve implant comprising an expandable framework and a plurality of valve leaflets disposed within the expandable framework; and
a filter element secured to the expandable framework and configured to be deployed downstream of the expandable framework, the filter element having a closed downstream end.

2. The replacement heart valve system of claim 1, wherein the filter element is configured to be deployed before the replacement heart valve implant.

3. The replacement heart valve system of claim 1, wherein the filter element is detachable from the expandable framework in situ.

4. The replacement heart valve system of claim 3, wherein the filter element is retrievable upstream through the replacement heart valve implant while leaving the replacement heart valve implant in place.

5. The replacement heart valve system of claim 3, wherein the filter element is retrievable downstream from the replacement heart valve implant while leaving the replacement heart valve implant in place.

6. The replacement heart valve system of claim 1, wherein the filter element includes an open mouth at an upstream end, wherein the open mouth is spaced apart from the expandable framework.

7. The replacement heart valve system of claim 1, wherein filter element is configured to capture particulates while permitting blood to pass through the filter element.

8. The replacement heart valve system of claim 1, further comprising a delivery sheath configured to transport the replacement heart valve implant and the filter element to a position upstream of a native heart valve.

9. A replacement heart valve system, comprising:
a filter element configured to be deployed across a native heart valve, wherein the filter element includes a lumen extending therethrough from a first end to a second end; and
a replacement heart valve implant comprising an expandable framework and a plurality of valve leaflets disposed within the expandable framework;
wherein the replacement heart valve implant is configured to be deployed within the lumen of the filter element.

10. The replacement heart valve system of claim 9, wherein the filter includes a medial portion having an hourglass shape in a deployed configuration.

11. The replacement heart valve system of claim 10, wherein the hourglass shape extends from the first end of the filter element to the second end of the filter element.

12. The replacement heart valve system of claim 9, wherein the first end includes a first sealing element configured to engage native tissue upstream of the native heart valve and the second end includes a second sealing element configured to engage native tissue downstream of the native heart valve.

13. The replacement heart valve system of claim 9, further comprising an expandable balloon disposed proximate a distal end of a balloon catheter, wherein the expandable balloon is configured to be expanded within the lumen of the filter element before deploying the replacement heart valve implant within the lumen of the filter element.

14. The replacement heart valve system of claim 9, wherein the filter element is permeable to blood.

15. A method of treating a native heart valve, comprising:
deploying a filter element across the native heart valve, wherein the filter element includes a lumen extending from a first end of the filter element to a second end of the filter element;
expanding an expandable balloon within the lumen of the filter element; and
thereafter, deploying a replacement heart valve implant within the lumen of the filter element.

16. The method of claim 15, wherein the filter element is configured to capture and retain debris dislodged while expanding the expandable balloon within the lumen of the filter element.

17. The method of claim 15, wherein the filter element includes a mesh extending from the first end to the second end.

18. The method of claim 15, wherein the filter element includes a permeable membrane extending from the first end to the second end.

19. The method of claim 15, expanding the expandable balloon within the lumen of the filter element includes urging the filter element radially outward against an annulus of the native heart valve.

20. The method of claim 15, wherein prior to deploying the replacement heart valve implant within the lumen of the filter element, the filter element permits native valve leaflets of the native heart valve to at least partially close the native heart valve.
